(19) United States
(12) Patent Application Publication
(10) Pub. No.: US 2010/0234737 A1
(43) Pub. Date: Sep. 16, 2010

(54) METHOD FOR ASSESSING SKIN IRRITATION USING INFRARED LIGHT

(76) Inventor: Miranda Aref Farage, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/402,541

(22) Filed: Mar. 12, 2009

(51) Int. Cl. A61B 6/00

(52) U.S. Cl. 600/473

(57) ABSTRACT

A method for determining skin irritation is provided. The method uses a source of infrared light to determine the skin temperature of an area of skin.

### FIELD OF THE INVENTION

This present invention relates to methods for assessing skin irritation using infrared light.

### BACKGROUND OF THE INVENTION

Testing is done to assess a personal care article's safety and effectiveness. One avenue of testing personal care articles, such as feminine hygiene pads or diapers, involves contacting the article with human skin. Many different tests are used to assess skin irritation caused by such contact. Examples of tests include standard occluded patch tests and tests that contact a chemical agent contained in personal care articles with skin. The skin contacted by the personal care article or chemical agent is then assessed to determine the presence of any effects such as irritation. Due to safety concerns, most personal care articles that will cause a strong or immediate skin reaction (irritation) are not tested on human subjects. Like other types of inflammation reactions, irritation of the skin triggers a series of events involving subsurface dilation of blood vessels, and an influx of inflammatory cells to the irritated area of skin. Irritated areas of skin often take on a red hue or coloration as compared to the un-irritated surrounding areas of skin. This irritation is visible on the surface, however early skin effects can be present but not visible on the surface of the skin.

Currently, determination of skin irritation on the surface of the skin requires physical examination, usually in the form of a visual assessment, of a patient by a skilled medical practitioner or expert skin grader. The reliance on a skilled practitioner has several disadvantages such as i) requires the services of a skilled medical practitioner and skilled technical staff; ii) methods used are time consuming and costly; iii) the diagnosis is subjective, and therefore not always reliable; iv) can only detect skin irritation after damage to the skin has already appeared on the surface.

For example, visual assessment of the genital area is usually done with a colposcope. A colposcope functions as a lighted binocular microscope to magnify the skin surface. However, when using visual assessment, discomfort is not always correlated with detectable physical symptoms. Thus, the absence of skin lesions or visual signs of irritation cannot be explained and sometimes cannot be clear.

It would be desirable to provide an objective and fairly rapid method for the determination of skin irritation, which does not require highly skilled personnel.

### SUMMARY OF THE INVENTION

A method for assessing skin irritation is provided. The method comprises the steps of providing a personal care article, which is contacted with an area of skin. Providing a source of infrared light and illuminating the area of skin with the source of infrared light to determine skin temperature.

A method for assessing skin irritation is provided. The method comprises the steps of providing a source of infrared light, which is used to illuminate an area of skin to determine a baseline skin temperature. Providing a personal care article and contacting the personal care article with the area of skin. Illuminating the area of skin with the source of infrared light to determine skin temperature and comparing the baseline skin temperature to the skin temperature after the area of skin has been contacted with the personal care article.

A method of assessing vulvovaginal skin irritation is provided. The method comprises the steps of providing a source of infrared light and illuminating a vulvovaginal area of skin with the infrared light to determine skin temperature.

### BRIEF DESCRIPTION OF THE DRAWINGS

FIG. 1 is a graph showing mean temperature measurements of upper arm test sites.

FIG. 2 is a graph showing mean visual scores of upper arm test sites.

FIG. 3 is a graph showing mean temperature measurements of upper arm test sites.

FIG. 4 is a graph showing mean afternoon visual scores of popliteal fossa test sites.

FIG. 5 is a graph showing mean afternoon temperature measurements of popliteal fossa test sites.

FIG. 6 is a graph showing mean morning visual scores of popliteal fossa test sites.

FIG. 7 is a graph showing mean morning temperature measurements of popliteal fossa test sites.

### DETAILED DESCRIPTION OF THE INVENTION

The present invention comprises a method for assessing skin irritation or effects. The method uses infrared light to detect increased skin temperature (increased over normal skin temperature of the area examined), which may be an indicator of skin irritation. The increase in skin temperature may then be evaluated, which in certain embodiments can lead to a determination of skin irritation.

As used herein, the term “personal care article” refers to a substrate or chemical agent that is applied to, or contacted with, a portion of the body such as the skin, hair, or teeth. For example personal care articles may be feminine care products (feminine hygiene pads, catamenial tampons, wipes, lotions), adult incontinence products, sanitary tissue products (facial tissue, toilet tissue, paper towels, wipes), baby care products (diapers, wipes), home care products (cleaning wipes, dusting wipes), beauty care products such as (shampoos, wipes) and oral care products (toothpaste, mouthwash).

The term “substrate” as used herein refers to woven material, non-woven material, or film material which is applied to, or contacted with, the body.

The term “irritation” as used herein, refers to an area of skin which exhibits indications of skin irritation such as erythema, inflammation, dryness, or innervation. The irritation may be visible on the skin surface or may not be visible on the skin surface (sub-surface irritation). In certain embodiments, irritation may be caused by chemical means or physical means.

The term “chemical means” as used herein, refers to a chemical agent which irritates an area of skin.

The term “physical means” as used herein, refers to a substrate that contacts an area of skin in such a manner as to cause irritation to the area of skin. Examples of physical means include tape stripping an area of skin, rubbing a substrate across an area of skin, and occluding an area of skin from the external environment.

The term “erythema” as used herein, refers to redness of the skin.

The term “dryness” as used herein, refers to powderiness or cracking of the skin.

The term “inflammation” as used herein, refers to a local response to cellular injury that is marked by capillary dilatation.

The term “innervation” as used herein, refers to the presence of neural tissue.

The term “vulvovaginal” encompasses the pudendal region of a human female, and includes anatomical sites such as the vulva, introitus, vagina, and cervix.

The term “vulvovaginal disorder” as used herein, refers to both common and uncommon conditions associated with vulvovaginitis, including vulvitis, vulvar vestibulitis syndrome, Lichen sclerosis, allergic contact dermatitis, systemic dermatoses, rare autoimmune diseases, and neuropathic vulvar pain syndromes and sensory symptoms (itch or pain, localized or generalized, provoked, intermittent or chronic).

The present invention is a reliable method for determining the presence of skin irritation or skin effects, such as reduction of dandruff or skin benefits from lotion or shampoo. The method uses infrared light to detect increased skin temperature in an area of skin (increased over normal skin temperature of the area of skin examined), which may be an indicator of skin irritation. Increased skin temperature can be an indicator of skin irritation, which may be visible on the skin surface or which may not be visible on the skin surface (sub-surface irritation), such as erythema, inflammation, dryness, or infection, where an increase in blood flow to the affected area, the release of pyrogens, or an increase in the metabolism of local leukocytes are responsible for localized increases in temperature.

In certain embodiments, the method of the present invention can be used to determine the presence of skin irritation in the vulvovaginal area. Skin irritation in the vulvovaginal may be caused by various factors, such as a vulvovaginal disorder or by a reaction to a personal care article, for example caused by mechanical friction or in response to a chemical component of the personal care article. The skin of the vulvovaginal area differs from the skin of the other areas of the body. The vulvovaginal area is moist, is limited in size, goes through cyclic changes such as temperature and pH, is difficult to access, has exposed and unexposed areas, and includes mucosal skin. All of these factors distinguish the vulvovaginal area from other portions of the body.

Infrared imaging has been used in the study of vascular disorders, arthritis, rheumatism, burn injuries, and dermatological disorders. Advances in infrared measuring devices have made it feasible to measure localized changes in skin temperature, such as those that may occur as a result of skin irritation. In certain embodiments of the present invention, infrared thermometers, such as the DermaTemp Infrared Thermographic Scanner from Exergen Co. of Watertown, Mass., measure skin temperature using blackbody radiation (generally infrared) emitted from objects. They are sometimes called laser thermometers if a laser is used to help aim the thermometer, or non-contact thermometers to describe the infrared thermometer's ability to measure temperature from a distance. By knowing the amount of infrared energy emitted by the object and its emissivity, the skin temperature can be determined.

The most basic design of an infrared thermometer consists of a lens to focus the infrared energy on to a detector, which converts the energy to an electrical signal that can be displayed in units of temperature after being compensated for ambient temperature variation. This configuration facilitates temperature measurement from a distance without having to contact the skin surface. As such, the infrared thermometer is useful for measuring temperature under circumstances where thermocouples or other probe type sensors cannot be used or do not produce accurate data for a variety of reasons.

As one skilled in the art will recognize, the methods described herein can be applied to various areas of the skin, or other areas of the body where skin irritation can be detected using infrared light, such as mucosal tissue in the mouth, or vulvovaginal area. However, in certain embodiments, the area of the skin includes forearm skin, upper arm skin, scalp or the skin at the back of the knee (popliteal fossa). It will be understood by one of ordinary skill in the art that areas of skin comprises both normal (healthy) skin, and skin which is abnormal or damaged, such as diseased, melanoma or burned skin.

In certain embodiments, irritation may be induced in an area of skin by chemical means or physical means. An example of the use of chemical means to irritate an area of skin may include contacting the area of skin with a skin irritant agent, for instance sodium lauryl sulfate and sodium laureth sulfate. An example of the use of physical means to irritate an area of skin may include tape stripping the area of skin. As known by one of skill in the art, tape stripping comprises applying a piece or pieces of tape to an area of skin, and then removing the tape such that the area of skin becomes irritated. Another example of physical means may include occluding an area of skin. One method of occluding comprises placing a patch, such as a Webril® patch, (Professional Medical Products Company) over an area of skin. Tape, such as an occlusive, hypoallergenic tape, such as Blenderm® tape, (3M Company) may be used to cover the patch and hold the patch in place on an area of skin. The patch may also comprise a chemical agent to facilitate irritation of the skin.

The methods of the present invention may be used to determine if the treatment of an area of skin with a formulation, such as a lotion or shampoo provides a benefit. For example, the methods of the present invention may include pretreatment (before being assessed using infrared light) or post-treatment (after being assessed using infrared light) of an area of skin with a lotion or shampoo. This would help determine whether a lotion or shampoo affects irritation on an area of skin. For example, an area of skin may be irritated, but before the area of skin is assessed with infrared light the area is pretreated with a lotion or shampoo. The area could then be compared to areas of skin that were irritated, but which did not receive any lotion or shampoo. Further, an area of skin may be irritated, and then assessed using infrared light after which the area is post-treated with a lotion or shampoo. The area of skin could then be assessed again using infrared light to determine if the lotion or shampoo had an effect on the irritation.

A lotion or shampoo may be in the form of emulsions or dispersions, and contain solids, gel structures, polymeric material, a multiplicity of phases (such as oily and water phase) and/or emulsified components. The lotion or shampoo may be shear thinning, or may strongly change viscosity around skin temperature to allow for transfer and easy spreading on a user's skin. The lotion or shampoo may sooth, moisturize, or lubricate a user's skin.

### EXAMPLES

Test participants (participants) for the tests varied in age between twenty-five (25) and fifty-eight (58), and had very sensitive to moderately sensitive skin (Types I-IV), as determined using Fitzpatrick's classifications, as disclosed in The Validity And Practicality Of Sun-Reactive Skin Types I Through VI. Archives Dermatology 1988 June; 124(6):869-71. Test sites on two areas of skin were tested, namely the upper arm and the skin area behind the knee (popliteal fossa). A test comprises application of test materials, patches or pads, to test sites on an area of skin, wherein the test sites are scored for erythema or measured for temperature.

In all tests, visual scoring of erythema at a test site was conducted by expert scorers under a 100 watt incandescent daylight bulb. Skin temperature measurement at a test site was also conducted by the expert scorers. The detection and assessment (scoring) of erythema was used in the tests as an indicator of skin irritation.

Skin erythema was scored using a scale “0” to “4” as set out below in Table 1.

TABLE 1
Erythema Scoring Scale

| | |
|---|---|
| 0 | No apparent cutaneous involvement |
| 0.5 | Faint, barely perceptible erythema |
| 1 | Faint, but definite erythema |
| 1.5 | Well-defined erythema |
| 2 | Moderate erythema; may have papules or deep fissures |
| 2.5 | Moderate erythema with barely perceptible edema; may have a few |
| | papules |
| 3 | Severe erythema (beet redness); may have generalized papules |
| 3.5 | Moderate-to-severe erythema with moderate edema |
| 4 | Moderate-to-severe erythema and/or extending edema, may have |
| | generalized vesicles or eschar formations |

Erythema was scored according to Table 1, where “0” is no apparent cutaneous involvement and “4” is moderate-to-severe spreading erythema or edema. If after scoring a test site exhibits an erythema score of “2” or higher, that test site receives no further treatment in the form of applied patches or pads. However, the test site is still scored until completion of the test. Any test site showing an erythema score of “2” or more at the final scoring time point was followed until the erythema score regressed to a “1.5” or less. For each patch or pad, the average for all erythema scores among the test sites on an area of skin is calculated for each completed grading day, and reported as the mean erythema score for that area of skin on that particular grading day.

The same scorer was used throughout a test, and the scorer was not aware of which patch or pad had been applied to a test site. Additionally, for the duration of a test a new patch or pad applied to a test site was the same as the patch or pad that was removed from the test site. For example, a patch containing 0.1% SLS would be replaced with another patch containing 0.1% SLS.

Skin temperature was measured using a DermaTemp® infrared thermographic scanner (Exergen Co., Watertown, Mass.). Temperature determinations were conducted at the same time as the visual scoring. Subjects underwent a 15 minute period of acclimation prior to the temperature measurement. Visual scoring and skin temperature measurements were conducted prior to sample application (i.e., at baseline).

### Example 1

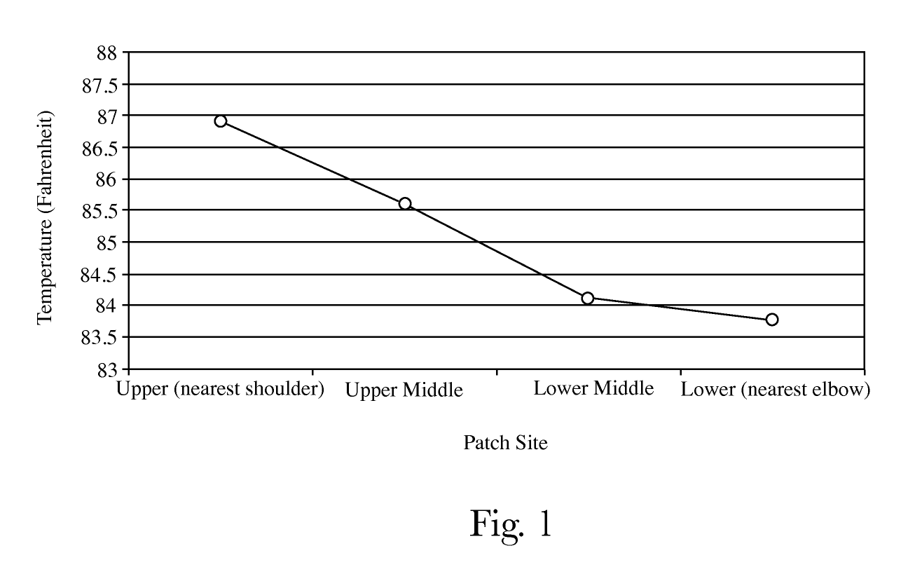

To determine baseline temperature variation along the length of the arm a DermaTemp® infrared thermographic scanner was used to measure the skin temperature of the sites of twenty (20) participants in which test patches would be placed on the arm—Upper (nearest shoulder), Upper Middle, Lower Middle, Lower (nearest elbow). The results, as shown in FIG. 1, demonstrate at the arm patch sites there was a consistent skin temperature variation along the arm, with the test sites nearest the shoulder having higher temperatures than those closer to the elbow. As a result of this observation, the temperature measurements in the arm patch tests were adjusted for patch site location when evaluating the results.

### Example 2

### 24 Hour Upper Arm Patch Tests

Each of the twenty (20) participants had four (4) test sites identified for the upper arm of each arm—Upper (nearest shoulder), Upper Middle, Lower Middle, and Lower (nearest elbow). Each test site is then demarcated on the lateral surface of the upper arm between the shoulder and elbow. Such demarcation can be done in manners known to one of ordinary skill in the art, such as marking the test sites with 0.5% Gentian violet to ensure that patches are applied to the same test sites each day for the duration of a test. Test sites are measured four (4) cm×four (4) cm, and with a minimum of two (2) cm between test sites. Patches applied at the test sites comprise an occlusive patch (Webril® patch Kendall LT, Chicopee Mass.) having placed thereon a square section measuring 0.7 inches per side taken from an Always®AF (wherein AF refers to an apertured film topsheet) feminine hygiene pad, Procter & Gamble Co., Cincinnati, Ohio or a Laurier® feminine hygiene pad Kao Co., Japan. Two control patches were also used, wherein an occluded patch contained 0.3 ml of saline (for a non-irritant control) or 0.1% SLS (for a positive irritant control). All patches were covered by an occlusive, hypoallergenic Blenderm® tape (3M, St. Paul Minn.). Participants were exposed to the test patches for about 24-hours per day for 4 consecutive days. Patches were removed by the participants thirty (30) to sixty (60) minutes prior to returning to the laboratory for scoring and reapplication of patches.

Before the patches were applied, a baseline mean erythema score using visual assessment and infrared light, using a DermaTemp® infrared thermographic scanner, was taken of each test site to which a patch was applied. To begin the study a patch was applied and then about twenty-four (24) hours after application, the patch was removed and the test site scored (24 h (hours)), using visual assessment and the temperature measured using a DermaTemp® infrared thermographic scanner. A new patch was then applied to the test site and the procedure repeated thrice more to produce three additional scores at time points 48 h, 72 h, and 96 h.

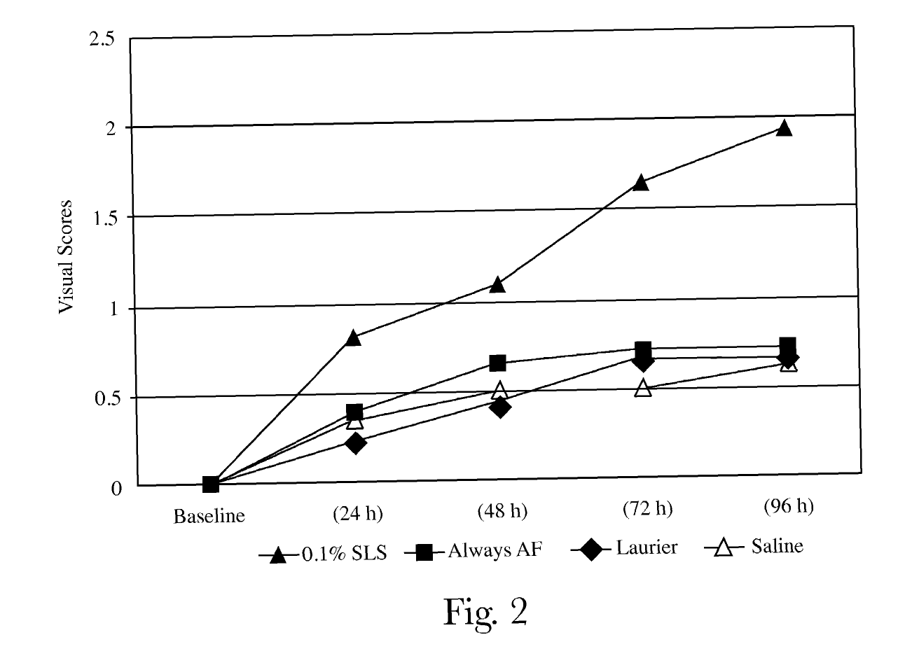

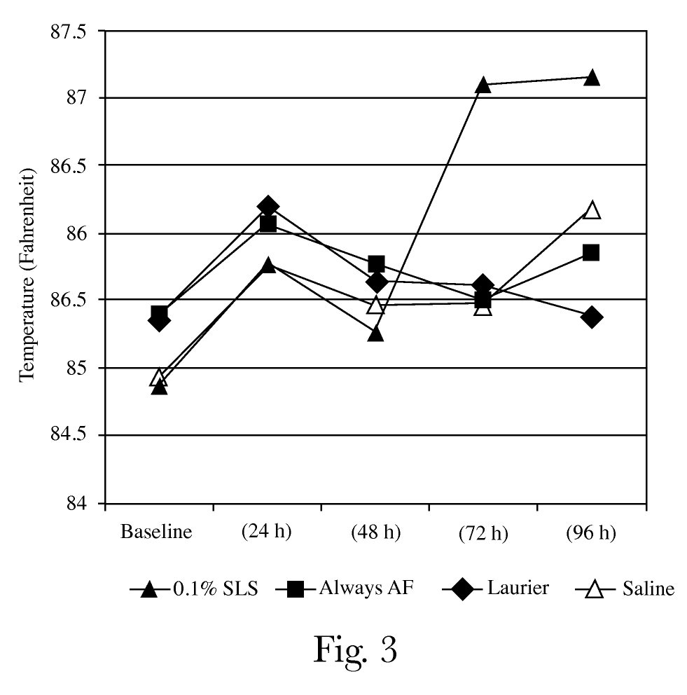

As shown in FIG. 2 and FIG. 3, the pattern of mean temperatures measured using the DermaTemp® infrared thermographic scanner were similar to the mean erythema scores resulting from the visual assessment.

Visual scores at the arm patch sites, as shown in FIG. 2, showed no differences between the mean scores produced by the patches including square sections taken from Always®AF and Laurier® and the patches including non-irritant control (saline). The patches including the positive irritant control (0.1% SLS) produced mean scores that were significantly higher than all other patches.

In FIG. 3, results of the skin temperature measurements were adjusted for patch site to compensate for the decrease in temperature as the test sites moved farther from the shoulder (Upper, Upper Middle, Lower Middle and Lower patch sites), as shown in FIG. 1. There was little difference between the mean skin temperatures resulting from square sections taken from Always®AF and Laurier® and the patches including non-irritant control (saline), as shown in FIG. 3. The irritant control (0.1% SLS) patch produced a mean temperature significantly different from the patches including square sections taken from Always®AF pads and Laurier® pads and the patches including non-irritant control (saline), after the final two patch applications (temperature measurements at 72 h and at 96 h). The results shown in FIG. 3 mirrored the results of the visual scoring of FIG. 2, wherein the irritant control (0.1% SLS) patch produced a higher mean temperature and higher mean visual score than the patches including square sections taken from Always®AF and Laurier® pads and the patches including non-irritant control (saline); demonstrating skin temperature measurements can be used to assess the presence (or absence) of skin irritation.

### Example 3

### Popliteal Fossa Tests

Test sites for the popliteal fossa were identified and then demarcated as stated in EXAMPLE 2. Test samples for the popliteal fossa test sites were Always®AF (wherein AF refers to an apertured film topsheet) feminine hygiene pads, Procter & Gamble Co., Cincinnati, Ohio and Laurier® feminine hygiene pads, Kao Co., Japan, or portions thereof. Always®AF and Laurier® pads were applied horizontally on the popliteal fossa test sites of twenty (20) participants, and held in place by an elastic knee band such as either Ace® Brand Knee Braces, Franklin Lakes, N.J., or Mueller Sport Care Elastic Knee Braces, Prairie du Sac, Wis. Each participant had an Always®AF pad positioned at the popliteal fossa test site of one leg and a Laurier® pad positioned at the popliteal fossa test site of the other leg. Participants were exposed to the test samples for 6-hours per day for 5 consecutive days. Pads were removed by the participants thirty (30) to sixty (60) minutes prior to returning to the laboratory for scoring and/or reapplication of materials.

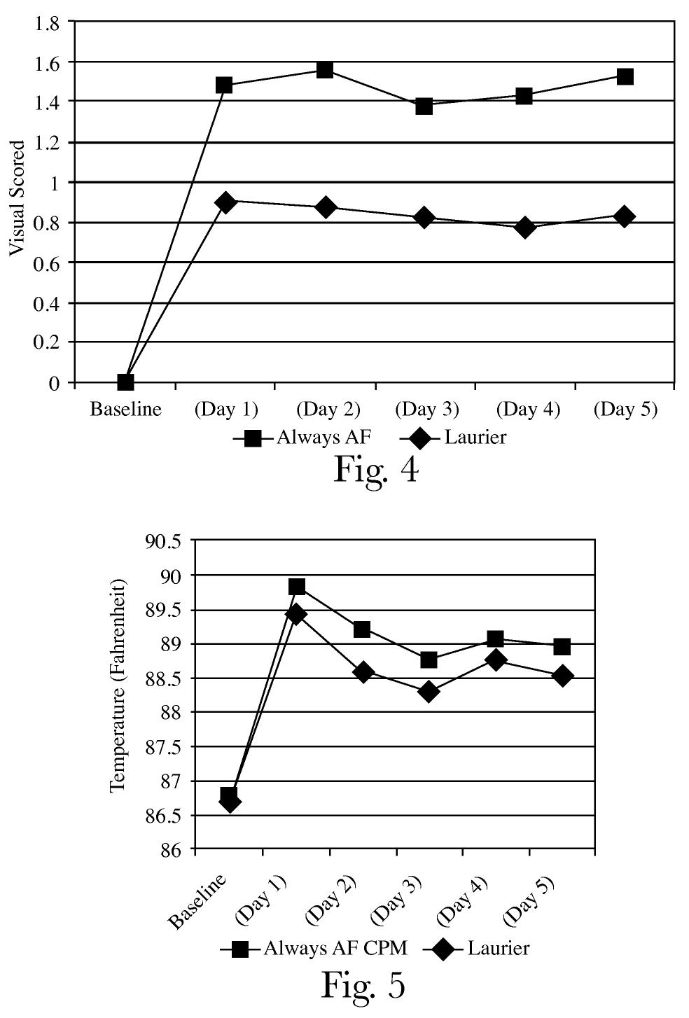

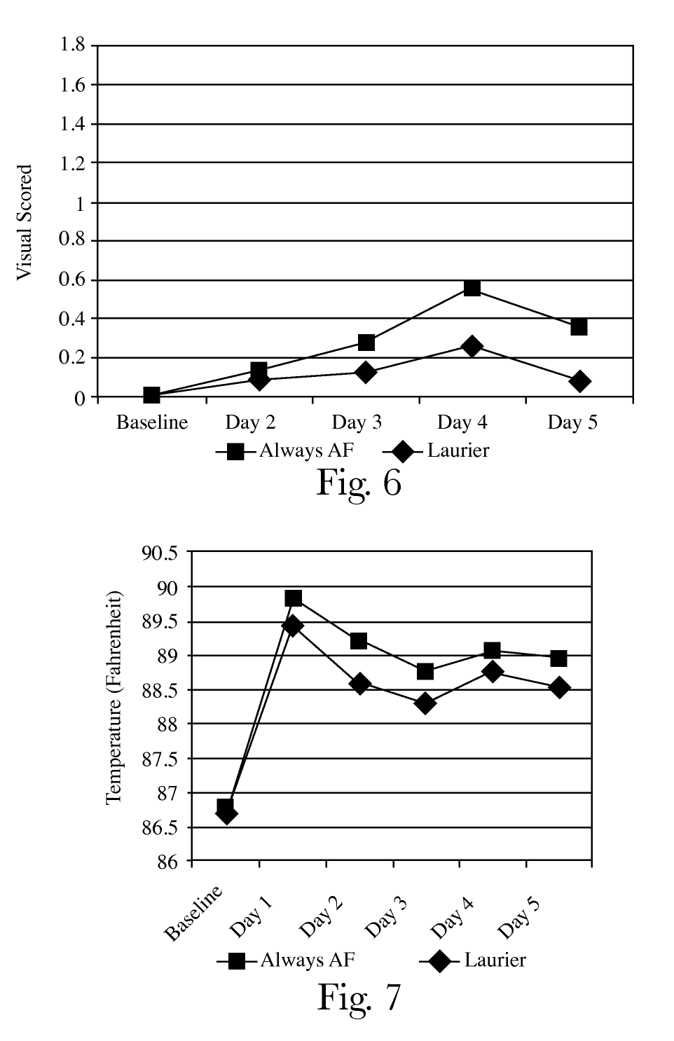

Visual assessment and skin temperature measurement, using a DermaTemp® infrared thermographic scanner, of each participant's popliteal fossa test sites was done at the start of the test (baseline, as described in EXAMPLE 2), 30-60 minutes after sample removal (afternoon grading), and the following mornings prior to each sample application (morning grading). Therefore, a first Always®AF pad or Laurier® pad was applied to a popliteal fossa test site for about six (6) hours then removed, and 30-60 minutes after sample removal the popliteal fossa test site was scored using visual assessment (FIG. 4—afternoon Day 1) and the skin temperature measured, using a DermaTemp® infrared thermographic scanner (FIG. 5—afternoon Day 1). The following morning before the application of a second Always®AF pad or Laurier® pad each popliteal fossa test site was scored using visual assessment (FIG. 6—morning Day 2) and the skin temperature measured again (FIG. 7—morning Day 2). After the morning visual scoring and skin temperature measurement an Always®AF pad and a Laurier® pad were applied to each popliteal fossa test site and worn by the participant for about six (6) hours. 30-60 minutes after sample removal the popliteal fossa test sites were scored using visual assessment (FIG. 4—afternoon Day 2) and the skin temperature measured (FIG. 5—afternoon Day 2). This process was repeated until the afternoon visual scoring (FIG. 4) and temperature measurement (FIG. 5) on Day 5. The afternoon visual scores (FIG. 4—Day 1 to Day 5) correlate with the afternoon temperature scores (FIG. 5—Day 1 to Day 5), as determined using a DermaTemp® infrared thermographic scanner. The Always®AF pads scored higher by visual assessment and by temperature measurement, using a DermaTemp® infrared thermographic scanner at all time points (FIGS. 4 and 5—Day 1 to Day 5) in the afternoon. The same was observed with the morning visual scores and temperature measurements (FIGS. 6 and 7—Day 1 to Day 5), with the exception of Day 4 where the visual score of the Always®AF pads were higher than the visual score of the Laurier® pads, but the temperatures as determined by infrared assessment, using a DermaTemp® infrared thermographic scanner were substantially the same. The results shown in FIGS. 4 to 7 demonstrate that skin temperature measurement is just as accurate for determining the presence of skin irritation as visual assessment, due to the substantial correlation of increased temperature (over baseline) and the visual determination of skin irritation.

As shown in FIG. 3, in the arm patch study, patches including Always®AF and Laurier® pad sections did not show differences in the mean temperature measurements at any time point. In contrast, a significant difference between Always®AF and Laurier® pads in the mean temperature measurements was observed when the pads were positioned in the popliteal fossa test sites, as shown in FIGS. 5 and 7 of EXAMPLE 3 (with the exception of the Day 4 morning temperature measurement). The difference between the temperature measurements of EXAMPLE 2 and EXAMPLE 3 involving the Always®AF and Laurier® pads is believed due to the differences in the test protocols. The arm patch test of EXAMPLE 2 only evaluates the inherent irritation due to the chemical composition of the sample; whereas the popliteal fossa test of EXAMPLE 3 evaluates an additional component of irritation, namely the mechanical irritation resulting from friction. This is due to the test site in EXAMPLE 3 being positioned at the popliteal fossa (area behind the knee), which causes the mechanical irritation by the pad being rubbed against the skin surface of the popliteal fossa when the knee is flexed. In contrast there is little or no movement of the skin surface against the Always®AF and Laurier® sections, when the test sites are positioned on the upper arm, as was done in EXAMPLE 2.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for assessing skin irritation comprising:
a. providing a personal care article;
b. contacting the personal care article with an area of skin;
c. providing a source of infrared light; and
d. illuminating an area of skin with the source of infrared light to determine skin temperature.

2. The method of claim 1 wherein an instrument provides the source of infrared light.

3. The method of claim 2 wherein the instrument is a DermaTemp infrared thermographic scanner.

4. The method of claim 1 wherein the personal care article comprises at least one of a feminine care product, adult incontinence product, sanitary tissue product, baby care product, home care product, beauty care product, or oral care product.

5. The method of claim 1 wherein the personal care article irritates the area of skin.

6. The method of 5 wherein the personal care article irritates the area of skin by at least one of chemical means or physical means.

7. The method of claim 1 wherein the area of skin includes at least one of forearm skin, upper arm skin, scalp and skin at the popliteal fossa.

8. The method of claim 1 wherein the area of skin is in the vulvovaginal region.

9. A method for assessing skin irritation comprising:
a. providing a source of infrared light;
b. illuminating an area of skin with the source of infrared light to determine a baseline skin temperature;
c. providing a personal care article;
d. contacting the personal care article with the area of skin;
e. illuminating the area of skin with the source of infrared light to determine skin temperature; and
f. comparing the baseline skin temperature to the skin temperature after the area of skin has been contacted with the personal care article.

10. The method of claim 9 wherein an instrument provides the source of infrared light.

11. The method of claim 10 wherein the instrument is a DermaTemp infrared thermographic scanner.

12. The method of claim 9 wherein the personal care article comprises at least one of a feminine care product, adult incontinence product, sanitary tissue product, baby care product, home care product, beauty care product, or oral care product.

13. The method of claim 9 wherein the personal care article irritates the area of skin.

14. The method of 13 wherein the personal care article irritates the area of skin by at least one of chemical means or physical means.

15. The method of claim 9 wherein the area of skin is treated with a lotion or shampoo before contacting the personal care article with the area of skin.

16. The method of claim 9 wherein the area of skin is treated with a lotion or shampoo after contacting the personal care article with the area of skin, but before illuminating the area of skin with the source of infrared light to determine skin temperature.

17. A method of assessing vulvovaginal skin irritation comprising:
a. providing a source of infrared light; and
b. illuminating a vulvovaginal area of skin with the infrared light to determine skin temperature.

18. The method of claim 17 wherein an instrument provides the source of infrared light.

19. The method of claim 18 wherein the instrument is a DermaTemp infrared thermographic scanner.

20. The method of claim 17 wherein the area of skin is treated with a lotion or shampoo.
